(19) United States
(12) Patent Application Publication
(10) Pub. No.: US 2017/0172960 A1
(43) Pub. Date: Jun. 22, 2017

(54) PHARMACEUTICAL FORMULATIONS FOR TREATING KIDNEY STONES AND METHODS FOR FABRICATING AND USING THEREOF

(71) Applicant: Imprimis Pharmaceuticals, Inc., San Diego, CA (US)

(72) Inventors: Dennis Elias Saadeh, Irvine, CA (US); Joseph S. Bitterman, San Diego, CA (US)

(21) Appl. No.: 15/383,211

(22) Filed: Dec. 19, 2016

Related U.S. Application Data
(60) Provisional application No. 62/271,020, filed on Dec. 22, 2015.
(60) Provisional application No. 62/272,894, filed on Dec. 30, 2015.

(51) Int. Cl. A61K 31/198; A61K 47/38; A61K 31/194

(52) U.S. Cl. CPC A61K 31/198; A61K 31/194; A61K 47/38

(57) ABSTRACT

Pharmaceutical compositions for treating, mitigating or preventing kidney stone disease, bladder stone disease or ureter stone disease are described, the compositions comprising a reducing agent capable of undergoing thiol-disulfide exchange with cystine to form a mixed disulfide and a citrate of an alkali metal or alkaline earth metal. Methods for fabricating the compositions and using them are also described.

### FIELD OF THE INVENTION

The present invention relates generally to the field of nephrology or urology, and more specifically to compositions and methods designed to treat, mitigate or prevent kidney stone disease, bladder stone disease, and ureter stone disease, and to methods of preparing such compositions.

### BACKGROUND

A significant portion of the population worldwide suffers from kidney stone disease or nephrolithiasis (as well as from related bladder and ureter stone diseases), which is a condition characterized by the appearance of stone-like matter (i.e., renal calculi, also known as nephroliths) that are formed and deposited in the patient's kidneys, bladder or ureter, respectively.

Typical renal calculi include those principally composed of calcium oxalate or phosphate, cystine (the stones formed as a result of a particular kind of nephrolithiasis, cystinuria), xanthine, uric acid and struvite. The symptoms include strong intermittent or constant pain (i.e., renal colic), hematuria, nausea, vomiting, and urinary urgency. In severe cases, nephrolithiasis can cause permanent kidney damage and even death.

Current non-invasive treatments include the use of α-blockers, pain relievers and hydration. Lithotripsy (breaking up stones using sound waves) is also widely used. All such treatments, however, are of limited effectiveness in many patients, particularly for larger stones. In many cases, surgical or ureteroscopical removal is the only viable option.

Accordingly, there exists a need for better methods and compositions for treatment, mitigation and/or prevention of nephrolithiasis and their symptoms. This patent specification discloses such pharmaceutical compositions that would achieve positive patient outcomes while being free of the drawbacks and deficiencies of existing formulations, and methods of fabricating and administering the same.

### BRIEF DESCRIPTION OF FIGURES

FIG. 1 demonstrates schematically a cross-section of the side view of an article of manufacture according to one embodiment of the invention.

FIG. 2 demonstrates schematically a cross-section of the side view of an article of manufacture according to another embodiment of the invention.

### SUMMARY

According to one embodiment of the invention, a pharmaceutical composition for treating, mitigating or preventing nephrolithiasis is provided, the composition comprising a therapeutically effective quantity of at least one pharmaceutically acceptable reducing agent capable of undergoing thiol-disulfide exchange with cystine to form a mixed disulfide, and a therapeutically effective quantity of at least one urine alkanizing agent selected from the group consisting of alkali metal salts of citric acid and alkaline-earth metal salts of citric acid.

According to other embodiments of the invention, a method for treating, mitigating or preventing kidney stone disease, bladder stone disease or ureter stone disease is provided, the method comprising administering to a patient in need thereof an above-mentioned pharmaceutical composition in the form of a pill, a powder, a tablet or a troche.

According to one embodiment of the invention a pharmaceutical article of manufacture is provided, the article comprising a first element that comprises the first component that includes at least one pharmaceutically acceptable reducing agent capable of undergoing thiol-disulfide exchange with cystine to form a mixed disulfide, and a second element that comprises the second component that includes at least one urine alkanizing agent selected from the group consisting of alkali metal salts of citric acid, alkaline-earth metal salts of citric acid, and sodium bicarbonate, wherein the first element is completely ensconced within the second element.

According to other embodiments of the invention, the first element can be a solid structure optionally coated with a pharmaceutically suitable coating, or can comprise an optionally acid resistant first solid shell defining a first space therein, the first space containing the first component. The second element can be a solid structure optionally coated with a pharmaceutically suitable coating, or can comprise an optionally acid resistant second solid shell, and the first element and the second element define the second space therebetween, wherein the second space contains the second component.

According to yet other embodiments of the invention, a method for treating, mitigating or preventing kidney stone disease, bladder stone disease or ureter stone disease, comprising administering to a patient in need thereof an above-mentioned pharmaceutical article of manufacture in the form of a pill, a capsule, a tablet or a troche.

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20.

The term “pharmaceutical composition” is defined as a chemical or biological compound or substance, or a mixture or combination of two or more such compounds or substances, intended for use in the medical diagnosis, cure, treatment, or prevention of disease or pathology.

The terms “kidney stone disease” and “nephrolithiasis” refer to a urological or nephrological disease or condition manifesting itself by having renal calculi (nephroliths) formed and deposited in the patient's kidneys.

The terms “bladder stone disease” and “ureter stone disease” refer to urological diseases or conditions manifesting themselves by having stone-like matter (cystoliths) formed and deposited in the patient's urinary bladder or ureter, respectively.

The term “cystinuria” refers to a kidney, bladder and/or ureter stone disease that is characterized by the formation of cystine stones in the kidneys, ureter, and bladder (i.e., the calculi formed as a result of precipitation of cystine out of urine).

The term “alkanizing agent” refers to a chemical compound or a drug that is administered to a patient having diseases or medical disorders associated with low pH of bodily fluids (e.g., blood), in order to increase the pH.

The term “reducing agent” refers to an electron-donor compound, i.e., a compound that donates an electron to another chemical species in a redox chemical reaction.

The terms “thiol” and “thiol moiety” refer to an organic compound that is a sulfur-containing analog of an alcohol, i.e., a compound containing the group —SH.

The term “thiol-disulfide exchange” refers to a chemical reaction described generally as follows:

RS—SR+R′SH [symbol] R′S—SR+RSH,

wherein each of R and R′ is an organic radical.

The terms “amino acid” and “amino acid moiety” refer to an organic compound having both a carboxyl (—COOH) and an amino (—NH₂) group.

The term “glycine” refers to aminoacetic acid having the structure NH₂—CH₂—COOH.

The term “cystine” refers to 2-amino-3-(2-amino-2-carboxy-ethyl)disulfanylpropanoic acid (i.e., an amino acid having the structure HOOC—CH(NH₂)—CH₂—S—S—CH₂—CH(NH₂)—COOH).

The term “citrate” refers to salts of citric acid (2-hydroxy-1,2,3-propanetricarboxylic acid).

The term “alkali metal” refers to the following elements of Group I of the Periodic Table: potassium, sodium, and lithium.

The term “alkaline-earth metal” refers to the following elements of Group II of the Periodic Table: magnesium, calcium, and barium.

The term “homogeneous mixture” refers to a combination of several separate substances forming a blend which visibly manifests itself as a single phase, where the individual components of the blend have the same proportions throughout a given volume creating a consistent mixture.

The terms “tablet” and “pill” refer to a generally spherical (for pills) or disk-shaped (for tablets) compressed solid articles containing a medicament to be taken orally.

The term “capsule” refers to a small, soluble container containing a dose of medicine, to be swallowed whole.

The term “troche” refers to a small tablet or lozenge (i.e., a medicated candy intended to be dissolved in the mouth), typically in a form of a disk, a ball or rhombic in cross-section, comprising medication and processed into a paste and dried.

The term “powder” refers to a pharmaceutical preparation in a solid dosage form comprised of a large number of finely divided solid particles of drugs or mixture of drugs and having the size of particles generally in the range of between about 0.1 μm and about 1 μm.

The term “therapeutically effective amount” is defined as the amount of the compound or pharmaceutical composition that will elicit the biological or medical response of a tissue, system, animal or human, that is being sought by the researcher, medical doctor or other clinician.

The term “pharmaceutically acceptable” is defined as a carrier, whether diluent or excipient, that is compatible with the other ingredients of the formulation and not deleterious to the recipient thereof.

The terms “administration of a composition” or “administering a composition” is defined to include an act of providing a compound of the invention or pharmaceutical composition to the subject in need of treatment.

### B. Embodiments of the Invention

According to embodiments of the present invention, pharmaceutical compositions are provided for treating, mitigating or preventing kidney stone disease, bladder stone disease or ureter stone disease. The compositions of the present invention comprise a therapeutically effective quantity of at least one pharmaceutically acceptable reducing agent capable of undergoing thiol-disulfide exchange with cystine to form a mixed disulfide, and a therapeutically effective quantity of at least one urine alkanizing agent selected from the group consisting of alkali metal salts of citric acid, alkaline-earth metal salts of citric acid, and sodium bicarbonate.

It is further specifically provided that the compositions of the invention are so formulated that the at least one reducing agent mentioned above and the at least one urine alkanizing agent also mentioned above form a homogeneous mixture, as the latter is defined herein.

In some embodiments, the reducing agent comprises a thiol moiety and an amino acid moiety and may be, e.g., N-(2-mercaptopropionyl) glycine having the chemical formula:

CH₃˜CH(SH)—C(O)—NH—CH₂—COOH,

also known as tiopronin, or under the trade name THIOLA® (Mission Pharmacal Co. of San Antonio, Tex.). Tiopronin is capable of binding cystine by thiol-disulfide exchange, to form a mixed disulfide of tiopronin-cysteine.

Alternatively, (2S)-2-amino-3-methyl-3-sulfanylbutanoic acid having the formula:

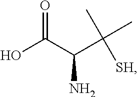

also known as D-penicilamine or under the trade name CUPRIMINE® (Valeant Pharmaceuticals International, Inc. Laval, Quebec, Canada) may be also used as the pharmaceutically acceptable reducing agent capable of undergoing thiol-disulfide exchange with cystine to form a mixed cysteine-containing disulfide. Penicilamine may be used as the sole reducing agent in the composition or in a combination with tiopronin, if desired. Another example of a reducing agent that may be used in addition to, or instead of, tiopronin and/or penicilamine is captopril (1-(3-mercapto-2-methyl-1-oxopropyl)-L-proline) known under the trade name CAPOTEN® (Bristol-Myers Squibb).

It is further specifically provided that the compositions of the invention are to be formulated as pills, tablets, capsules or troches for oral administration.

The concentration of the reducing agent(s) described above, in the compositions may be between about 25.0 mass % and about 50.0 mass % of the total mass of the pill, tablet, capsule, troche or powder. In other words, for a typical pill, tablet, capsule, troche or powder having the total mass of between 400 mg and about 2.0 g, the mass quantity of the reducing agent(s) may be between about 100 mg and about 1,000 mg, such as between about 150 mg and about 500 mg, for example about 200 mg.

With respect to the second active component of the pharmaceutical compositions of the present invention, a urine alkanizing agent, such as potassium citrate, sodium citrate, magnesium citrate, sodium bicarbonate or combinations thereof may be used.

The concentration of the urine alkanizing agent(s) described above in the compositions may be between about 25.0 mass % and about 70.0 mass % of the total mass of the pill, tablet, capsule, troche or powder, for example, about 60.0 mass %. In other words, for a typical pill, tablet, capsule, troche or powder having the total mass of between about 400 mg and about 2.0 g, the mass quantity of the urine alkanizing agent(s) may be between about 100 mg and about 800 mg, such as between about 200 mg and about 800 mg, for example about 500 mg.

As mentioned above, the pharmaceutical composition may further optionally include one or several pharmaceutically acceptable excipient(s). In some embodiments, an excipient that can be used may be one or several filler(s) to be selected by those having ordinary skill in the art, such as microcrystalline cellulose and/or hydroxypropyl methylcellulose (e.g., Methocell® E4M or Methocell® K100 available from Dow Chemical Co. of Midland, Mich.). For example, as is known in the art, Methocell® E4M, which is a component allowing delayed release, can be used for preparing the formulations in the form of AR (i.e., acid-resistant) capsules to protect from gastric acid and delay dissolution. Therefore, in some embodiments, formulations may be optionally compounded as delayed release compositions.

The concentration of such excipient(s), if used, in the compositions may between about 20.0 mass % and about 25.0 mass % of the total mass of the pill, tablet, capsule, troche or powder. In other words, for a typical pill, tablet, capsule, troche or powder having the total mass of between about 400 mg and about 2.0 g, the mass quantity of the urine alkanizing agent(s) may be between about 100 mg and about 400 mg.

In some other embodiments, one or both of the reducing agent(s) and urine alkanizing agent(s) may be utilized without the use of encapsulating shells; instead uncoated or coated tablets, pills or troches may be employed. If the coated tablets, pills or troches are used, those having ordinary skill in the art will select the most appropriate coatings, as is known in the art. Acid-resistant and/or delayed release coatings may be so used, if desired.

According to further embodiments, methods for fabricating the above-described pharmaceutical compositions are provided. A one-batch formulation method may be used, where the components of the pharmaceutical formulation can be combined in single container; the components may be added to the container simultaneously or consecutively. In one exemplary, non-limiting procedure, a quantity of reducing agent(s) and a quantity of urine alkanizing agent(s) may be placed into a mixing container (e.g., a mortar) followed by dry mixing with a pestle.

The resulting product may then be adapted for oral administration, for example formulated and shaped as pill, tablet, capsule, troche or powder according to methods known to those having ordinary skill in the art. The medication prepared as described above may then be prescribed and given to a patient for treating, mitigating or preventing kidney stone disease, bladder stone disease or ureter stone disease. Among various kinds of kidney, bladder or ureter stone disease that may be treated, one kind of treatment that is particularly envisioned according to embodiments of the present invention is the treatment, mitigation or prevention of cystinuria.

According to still further embodiments of the invention, pharmaceutical articles of manufacture are provided. Each article comprises a first element, comprising the first component that includes at least one pharmaceutically acceptable reducing agent capable of undergoing thiol-disulfide exchange with cystine to form a mixed disulfide. The article further provides a second element, comprising the second component that includes at least one urine alkanizing agent selected from the group consisting of alkali metal salts of citric acid, alkaline-earth metal salts of citric acid, and sodium bicarbonate. The first element is incorporated into the second element, so that the former is completely ensconced within the latter.

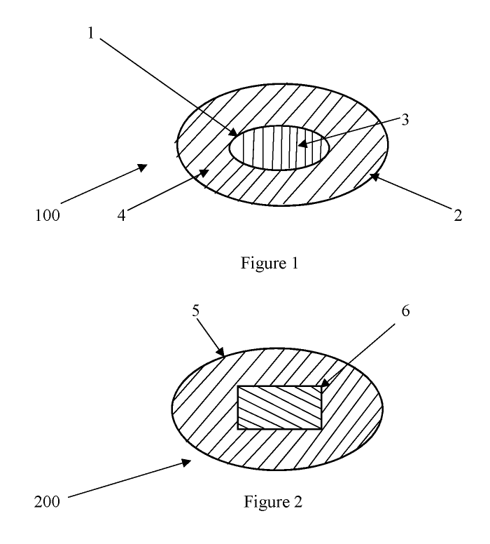

Some of such pharmaceutical articles of manufacture are illustrated by FIGS. 1 and 2. For example, FIG. 1 shows a cross-section of the side view of an article 100 (“capsule-in-capsule”) having an inner capsule 1 incorporated into a large outer capsule 2. The space 3 inside capsule 1 is filled with a quantity of one or several reducing agent(s) capable of undergoing thiol-disulfide exchange with cystine to form a mixed disulfide, as described above. The space 4 between capsules 1 and 2 is filled with one or several urine alkanizing agent(s) also described above (e.g., alkali metal salts of citric acid, alkaline-earth metal salts of citric acid, sodium bicarbonate).

Those having ordinary skill in the art can select the most appropriate sizes for capsules 1 and 2. As a general guidance only, the longer diameter of the larger capsule 2 can be between about 20 mm and about 22 mm, such as between about 15 mm and about 20 mm, for example, about 20 mm, and the shorter diameter of the larger capsule 2 can be between about 8 mm and about 12 mm, for example, about 10 mm. The dimensions of the smaller inner capsule 1 may be generally at about 50% of the corresponding dimensions of the outer capsule 2.

Various other embodiments are envisioned having similar combined pharmaceutical articles, e.g., as shown by the side view of a cross section of one such article as represented by FIG. 2. As shown in FIG. 2, the article 200 includes a larger capsule 5 incorporating a smaller tablet 6 made of one or several reducing agent(s) described above. The rest of the capsule 5 is filled with one or several urine alkanizing agent(s) also described above. Another illustrative, non-limiting example (not shown) can be an article having a larger tablet made of one or several urine alkanizing agent(s) incorporating a smaller tablet made of one or several reducing agent(s).

It will be understood by those having ordinary skill in the art that the specific dose levels and frequency of administration for any particular patient may be varied and will depend upon a variety of factors including the activity of the specific compound employed, the metabolic stability and length of action of that compound, the age, body weight, general health, gender, diet, and the severity of the particular kidney, bladder and/or ureter stone disease being treated.

In additional embodiments, pharmaceutical kits are provided. The kit includes a sealed container approved for the storage of pharmaceutical compositions, and the above-described pharmaceutical composition. An instruction for the use of the composition and the information about the composition are to be included in the kit.

The following examples are provided to further elucidate the advantages and features of the present invention, but are not intended to limit the scope of the invention. The examples are for the illustrative purposes only. USP pharmaceutical grade products were used in preparing the formulations described below.

### Example 1. Preparing a Pharmaceutical Composition No. 1

A pharmaceutical composition can be prepared as described below. The following components were used in the amounts and concentrations specified:

(1) about 200.0 mg of tiopronin powder;

(2) about 500.0 mg of potassium citrate powder; and

(3) about 100 mg of Methocell® E4M powder.

Tiopronin, potassium citrate, and Methocell® E4M powders can be mixed using a mortar and pestle method by using the principles of trituration and geometric dilution known to those having the skill in the art of preparing pharmaceutical compositions. To wit, potassium citrate, and Methocell® E4M powders can be mixed into tiopronin powder in small portions until a completely homogenous mixture has been obtained.

The resulting product can be encapsulated into AR Caps® Clear, Size 0 or 1, the capsules can be put into an airtight container, and the container can be labeled accordingly.

Although the invention has been described with reference to the above examples, it will be understood that modifications and variations are encompassed within the spirit and scope of the invention. Accordingly, the invention is limited only by the following claims.

We claim:

1. A pharmaceutical composition for treating, mitigating or preventing kidney stone disease, bladder stone disease or ureter stone disease, the composition comprising:
(a) a therapeutically effective quantity of a first component, wherein the first component comprises at least one pharmaceutically acceptable reducing agent capable of undergoing thiol-disulfide exchange with cystine to form a mixed disulfide; and
(b) a therapeutically effective quantity of a second component, wherein the second component comprises a therapeutically effective quantity of at least one urine alkanizing agent selected from the group consisting of alkali metal salts of citric acid, alkaline-earth metal salts of citric acid, and sodium bicarbonate,
wherein the first component and the second component form a homogeneous mixture.

2. The composition of claim 1, wherein the reducing agent comprises a thiol moiety and an amino acid moiety.

3. The composition of claim 2, wherein the amino acid is glycine.

4. The composition of claim 1, wherein the reducing agent is selected from the group consisting of tiopronin, penicilamine, and captopril.

5. The composition of claim 4, wherein the reducing agent is tiopronin.

6. The composition of claim 1, wherein the alkali or alkaline-earth metal salts of citric acid are selected from the group consisting of potassium citrate, sodium citrate, and magnesium citrate.

7. The composition of claim 6, wherein the urine alkanizing agent is potassium citrate.

8. The composition of claim 1, wherein the composition is in a form selected from the group consisting of a pill, a tablet, powder, a capsule, and a troche.

9. The composition of claim 1, further comprising a third component, wherein the third component provides the composition with a delayed release feature.

10. The composition of claim 9, wherein the third component is hydroxypropyl methylcellulose.

11. A method for treating, mitigating or preventing kidney stone disease, bladder stone disease or ureter stone disease, comprising administering to a patient in need thereof the pharmaceutical composition of claim 1, thereby treating, mitigating or preventing kidney stone disease, bladder stone disease or ureter stone disease.

12. The method of claim 11, wherein the reducing agent comprises a thiol moiety and an amino acid moiety.

13. The method of claim 12, wherein the amino acid is glycine.

14. The method of claim 11, wherein the reducing agent is selected from the group consisting of tiopronin, penicilamine, and captopril.

15. The method of claim 14, wherein the reducing agent is tiopronin.

16. The method of claim 11, wherein the alkali or alkaline-earth metal salts of citric acid are selected from the group consisting of potassium citrate, sodium citrate, and magnesium citrate.

17. The method of claim 16, wherein the urine alkanizing agent is potassium citrate.

18. The method of claim 11, wherein the composition is in a form selected from the group consisting of a pill, a tablet, powder, a capsule, and a troche.

19. The method of claim 11, wherein the disease is cystinuria.

20. A pharmaceutical article of manufacture, comprising the composition of claim 1, and further comprising:
(a) a first element, comprising the first component; and
(b) a second element, comprising the second component,
wherein the first element is completely ensconced within the second element, with the further provisos that
(1) the first element is a solid structure optionally coated with a pharmaceutically suitable coating or the first element comprises an optionally acid resistant first solid shell defining a first space therein; and
(2) the second element is a solid structure optionally coated with a pharmaceutically suitable coating, or the second element comprises an optionally acid resistant second solid shell, and the first element and the second element define the second space therebetween, wherein the second space contains the second component.
